(19) United States
(12) Patent Application Publication
(10) Pub. No.: US 2022/0071330 A1
(43) Pub. Date: Mar. 10, 2022

(54) Hair Extension Device and Method

(71) Applicants: Erin Marie Tedesco, Katonah, NY (US); Andrea Mason, Green Island, NY (US)

(72) Inventors: Erin Marie Tedesco, Katonah, NY (US); Andrea Mason, Green Island, NY (US)

(21) Appl. No.: 17/250,581

(22) PCT Filed: Aug. 6, 2019
(86) PCT No.: PCT/US19/45284

§ 371 Date: Apr. 23, 2021

Related U.S. Application Data
(60) Provisional application No. 62/764,517, filed on Aug. 7, 2018.

(51) Int. Cl. A41G 5/00; A61K 8/37; A61K 8/34; A61K 8/87; A61Q 5/06

(52) U.S. Cl. CPC A41G 5/008; A61K 8/37; A61Q 5/06; A61K 8/87; A61K 8/34

(57) ABSTRACT

A removable device for temporary attachment to the head of a person having native hair, said device comprising a base and a substrate, said base comprising a top surface and a bottom surface and a plurality of nonnative hair pieces secured thereto and extending outward from said top surface, said base having a generally crescent shape comprising a center portion and at least two side portions extending outward from said center portion, said side portions aligned generally parallel to one another when attached to the head of a person, said substrate comprising a top and a bottom surface having a shape similar to at least a portion of the shape of said base, and at least one of said bottom surface of said base and said top surface of said substrate adapted to receive an adhesive.

### FIELD OF THE INVENTION

The invention relates to devices and methods for attaching non-native hair to the head of a person in need thereof.

### BACKGROUND

The following describes a device worn on the head of a person in need thereof to give the wearer the appearance of a natural head of hair, and in particular a natural hair part. There are known devices, referred to in the art for example as wigs, hair extensions and hair pieces, that are used primarily by those lacking enough natural hair to effectively cover their scalp, revealing either areas of baldness or patches of hair thin enough to reveal the scalp surface through the remaining natural hair. Such devices are also known to be used by persons seeking to add non-native hair to their own native hair to give the impression of having thicker or longer hair or to achieve a certain desired hairstyle. However, the devices known in the art do not provide a solution to those seeking a lightweight, removable, adhesively secured device mounted primarily on the top of the head to give the impression of a natural hair part. For certain users of hair extension devices, the current solutions provide means of attaching hair devices by sewing in or connecting comb-like attachments on the device to existing hair on their head. However, for others, in particular for women with thinning hair at the top of their heads (female pattern baldness), the only option with current devices is to shave the remaining hair on the top of their heads and attach a full hair piece with adhesives applied directly to the scalp.

For example, U.S. Pat. No. 9,486,024 describes a hair extension system wherein hair sections are connected to a track that allows for the hair to be moveable along the track. U.S. Pat. No. 4,784,713 describes an apparatus and method for forming hair pieces that provides for attaching hair to a membrane and cutting to a desired length. U.S. Pat. No. 7,201,171 discloses a hair extension system containing an adhesive strip for fastening. U.S. Patent Publication 2004/0079382 discloses a hair piece having a base portion and a plurality of hairs and structure for attaching the hair piece to the head of the user. The hair piece is said to have a slit that maintains the natural appearance of the parts in the user's hair. U.S. Patent Publication 2010/0000567 discloses a U-shaped closure comprising a top hair net and top decorative hair attached to the top hair net. U.S. Patent Publication No. 2012/0174939 discloses a detachable, self adhesive, multipiece wiglet and anchor cap system. The system is said to be made up of individual shapes and sizes of wiglets. US Patent Publication 2013/0098380 describes a hair piece for creating a hair part which is said to include a base having an outer perimeter, which includes a front edge, a back edge opposite the front edge a first side and a second side. The base is also said to include a slit extending from the front edge toward the back edge and terminating at an apex spaced inwardly from the back edge such that the apex is positioned between the front and back edges. Finally, GB2327605A describes a hair extension in the form of a scalp patch comprising a flexible sheet substrate, e.g. of polyurethane, bearing hair on one surface and having a marginal area devoid of hair whereby the patch may be sewn onto cornrows braided from the subject's natural hair. The publication states the hair may have a parting or crown and the patch is adapted to cover the top of the head as part of a hair extension method in which wefts of hair are attached to cornrows formed lower on the head.

The devices currently known in the art, including those cited above, are deficient in that they do not provide a light weight hair extension alternative that can be easily installed and removed, utilizing a wearer's own native hair and comprising light-weight materials and adhesives, that allows for application to the top portion of the wearer's head to provide a natural looking hair part. In particular the art does not solve the problem of requiring the user to manipulate their remaining native hair, for example by forming into cornrows to allow for sewing of the device to the hair or shaving the head to allow for attachment of the device directly to the scalp. In contrast, the hair extension device of the invention can be easily installed and removed, is lightweight and malleable, remains fixed in place on the head during use without the need for stitching of the device to existing hair and is specifically adapted for those seeking to recreate a natural looking hair part. Further, with the use of this invention, there would be no need for sprays or heavy and uncomfortable hair weaves or wigs. As described more fully below, this invention is intended to be secured with a light adhesive that can be released from the native hair by spraying a solvent that gently reverses the adhesive action, without pulling or breaking the user's native hair. The invention is intended to be reusable, easy to clean, lightweight and specifically made to be invisible at the root of the hair part and crown to add volume and give women the confidence to wear ones real hair.

References referred to herein, though not admitted to being prior art, are hereby incorporated by reference in their entirety by reference thereto.

### SUMMARY OF THE INVENTION

Thus, the invention provides a removable device for temporary attachment to the head of a person having native hair, said device comprising a base and a substrate, said base comprising a top surface and a bottom surface and a plurality of nonnative hair pieces secured thereto and extending outward from said top surface, said base having a generally crescent shape comprising a center portion and at least two side portions extending outward from said center portion, said side portions aligned generally parallel to one another when attached to the head of a person, said substrate comprising a top and a bottom surface having a shape similar to at least a portion of the shape of said base, and at least one of said bottom surface of said base and said top surface of said substrate adapted to receive an adhesive.

The invention also provides a hair extension device for attachment to the hair on the head of a person at or above the crown of the head, said device comprising a base and a substrate, said base comprising a plurality of hair pieces attached thereto, said base having a generally crescent shape comprising at least two side portions extending outward from a single curved region, said side portions adapted to be aligned generally parallel to one another when attached to the hair on the head of a person, said substrate having a shape substantially identical to the shape of at least a portion of the base.

The invention further provides a method of attaching a hair extension device comprising non-native hair to the native hair on the head of a person in need thereof, wherein the device comprises a substrate, a base and a plurality of nonnative hair pieces attached to said base, and an adhesive composition, said base having a generally crescent shape comprising at least two connected side portions extending from a central region and non-native hair attached thereto, and said substrate comprising a shape substantially similar to at least a portion of said base, wherein the method comprises aligning a portion of the native hair of the person between the base and the substrate and subsequently affixing the base and substrate together by contacting the substrate, base and native hair together with the adhesive, whereby the hair extension device is attached to the native hair on the head of the person.

The invention also provides a method of creating a natural looking hair part on the head of a person in need thereof, said person having native hair, wherein the device comprises a substrate, a base and a plurality of nonnative hair pieces attached to said base, at least one of said substrate and said base comprising an adhesive composition attached thereto, said base having a generally crescent shape comprising at least two connected side portions extending outward from a central region, and said substrate comprising a shape substantially similar to said base, said method comprising aligning the side portions generally parallel to one another, distributing between the base and the substrate native hair from a portion of the person's head at or above the crown of the head, and contacting the base and substrate in a manner such that the base and substrate are affixed to the native hair by way of the adhesive, distributing additional native hair over the side portions and comingling said native hair with the nonnative hair from the device, thereby creating a natural looking hair part.

The invention further provides a kit for use in attaching a hair extension device to the native hair on the head of a person in need thereof, wherein the kit comprises a hair extension device, said device comprises a substrate, a base and a plurality of nonnative hair pieces attached to said base, said base having a generally crescent shape comprising at least two connected side portions extending outward from each other, and said substrate comprising a shape substantially similar to said substrate, and instructions for attaching the hair extension device to the native hair on the head of the person.

These and other embodiments and benefits of the invention will be more fully set forth in the detailed description set forth below and the drawing figures attached hereto.

### BRIEF DESCRIPTION OF THE DRAWINGS

FIG. 1 is perspective view of an embodiment of the device of the invention showing the substrate and the base where the base comprises a single piece.

FIG. 2 is a perspective view of an embodiment of the device of the invention showing the substrate and the base where the base comprises a plurality of pieces.

FIGS. 3A-3C are side views of certain embodiments of the device of the invention; FIG. 3A shows the side view of the device where the base comprises a single piece;

FIG. 3B shows the side view of the device where the base comprises a plurality of pieces; FIG. 3C shows the side view of the device where the base comprises a single piece and depicts an embodiment of the invention where the substrate and base both contain an adhesive layer.

FIG. 4 is a perspective view of an embodiment of the device of the invention showing how native hair is placed between the substrate and the base in use.

FIG. 5 is a perspective view of an embodiment of the device during the method of installation, depicting a view from the side of the head of the wearer where the non-native hair on the device is integrated with the native hair of the wearer.

FIG. 6 is a perspective view of an embodiment of the device during the method of installation, depicting a view from the top of the head of the wearer where the non-native hair on the device is integrated with the native hair of the wearer.

FIG. 7 is a perspective view of an embodiment of the device during the method of installation, depicting a view from the top of the head of the wearer where the device is installed with the side portions of the substrate and corresponding portions of the base are applied substantially parallel to each other respectively to provide a hair part that is off center of the top of the head of the wearer.

FIG. 8 is a perspective view of an embodiment of the device during the method of installation, depicting a view from the top of the head of the wearer where the device is installed with the side portions of the substrate and corresponding portions of the base are applied substantially parallel to each other respectively to provide a hair part that is generally at the center of the top of the head of the wearer.

### DETAILED DESCRIPTION

The invention will now be further described with reference to the attached drawings.

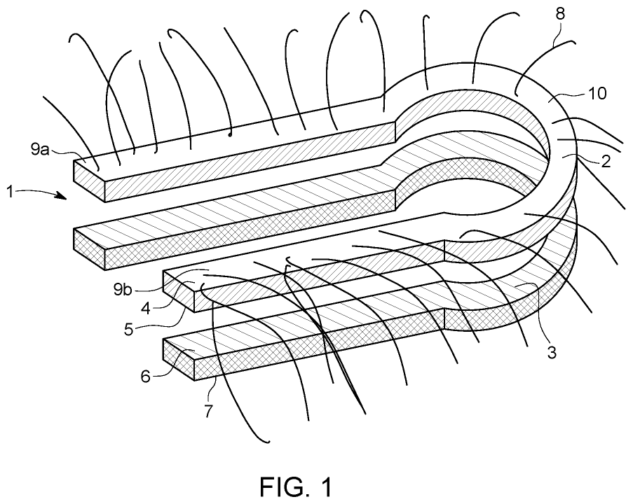

An embodiment of the invention is shown in FIG. 1, where the removable device 1 comprising a base 2 and a substrate 3, said base comprising a top surface 4 and a bottom surface 5 and a plurality of nonnative hair pieces 8 secured thereto and extending outward from said top surface 4. In certain embodiments of the invention the base will comprise a generally crescent shape comprising a center portion 10 and at least two side portions 9a and 9b extending outward from said center portion, said side portions depicted as aligned generally parallel to one another as would be seen when attached to the head of a person in order to approximate a natural hair part. In certain embodiments the crescent shape can also be described as keyhole shape when side portions 9a and 9b are aligned generally parallel to one another with center portion 10 defining a generally circular opening surrounded by the shape of the base 2 and the opening defined between side portions 9a and 9b when aligned generally parallel having a more narrow, generally rectangular shape. The substrate comprises a top surface 6 and a bottom surface 7 and has a shape similar to the shape of the base. In certain embodiments, the shape of the substrate will be substantially identical to the shape of the base. In certain embodiments at least one of said bottom surface 5 of said base and said top surface 6 of said substrate will be adapted to receive an adhesive.

In certain embodiments the substrate and the base comprise one or more support materials. As used herein, the base and substrate of the invention do not necessarily comprise the same support materials but can be varied according to desired compatibility with, for example, choices of non-native hair, method of attaching non-native hair to base, particular adhesive composition to be applied, and desired size and/or weight of device. In certain embodiments, the substrate and/or base comprise a support material comprising natural materials. As used herein, natural materials can include, for example, fibers containing plant materials such as cellulose fibers, non-limiting examples of which include hemp, flax, ramie, jute, harakeke, sisal, alfa, cotton, coir, silk, feather, bamboo and wool. In certain embodiments, the substrate and/or base comprise a support material comprising non-natural materials. As used herein the non-natural materials may comprise fibers obtained through chemical manufacture, nonlimiting examples of which include fibers comprising polyester, polyamide, and polypropylene materials, as well as inorganic materials, including such materials containing silicon and boron. In certain embodiments the support materials may comprise hydrocolloid or hydrogel material. In certain embodiments the support materials may also comprise an elastomer film, such as a film comprising one or more of polyethylene, polyvinyl chloride, ethylene-vinyl acetate copolymer, polyamide, polyester, polyurethane, acrylic polymer and the like. Of such non-natural materials, preferred materials are those presently known or determined to be safe for use in application on or near the body of an animal, including humans, such as for surgical and medical device applications. In certain embodiments the materials can comprise fibers which can be multifilament or monofilament materials or a combination thereof. In certain embodiments, the substrate and/or base can contain a mixture of natural and non-natural materials. In certain embodiments the support materials are woven, such as in a mesh fabric. In certain embodiments the device of the invention comprises a support material or combination of support materials having a weight ranging from at least about 5 g/m² (gsm) to about 200 gsm. In additional embodiments the support materials can comprise a material having a weight ranging from at least about 10 gsm to about 150 gsm, from at least about 20 gsm to about 100 gsm, and from at least about 30 gsm to about 50 gsm. In certain embodiments the support material can comprise so called “shaping fabrics”, which comprise for example fibers such as nylon, rayon, Lycra, Spandex and various combinations thereof, including combinations with other natural and non-natural materials. Examples of such fabrics are disclosed in U.S. Pat. Nos. 9,930,916, 9,801,420, 7,024,892, and 6,267,176. In certain embodiments, the support and base comprise the same materials. In certain embodiments the support and base comprise different materials. In certain embodiments comprising fiber materials, the fibers can comprise a single fiber comprising a mixture of materials. In certain embodiments comprising fiber materials, the substrate and/or base can comprise a mixture of fibers, each fiber comprising a single type of material. In certain embodiments the substrate and base comprise a color or tint that matches the non-native hair attached to the base or the native hair of the wearer, such that the base and substrate materials are not visible once fully installed on the wearer.

Methods of attaching non-native hair to the base to produce the device of the invention can be done by means known in the art, including weaving the non-native hair to the support materials or attaching by other means such as adhesives. Methods of attaching non-native hair to a wig device are described, for example in U.S. Pat. Nos. 4,784,713, 5,921,253, 6,446,634, U.S. Patent Publication No. 2004/0225314 and EP1832188A1. Non-native hair may also be incorporated into the materials of which the base is comprised as part of the manufacture of the base material, such as by coextrusion or incorporation into a matrix. As used herein the term “non-native” refers to all types of materials typically used for production of wigs and hair pieces, including synthetic and natural fibers, and materials that can approximate the look and feel of natural hair, as well as human and other animal hair. Non-native hair is meant to include human hair, including hair of the intended wearer that is not actually attached to the wearer's head. In contrast, as used herein, “native hair” includes all or any portion of the hair attached to the wearer's head.

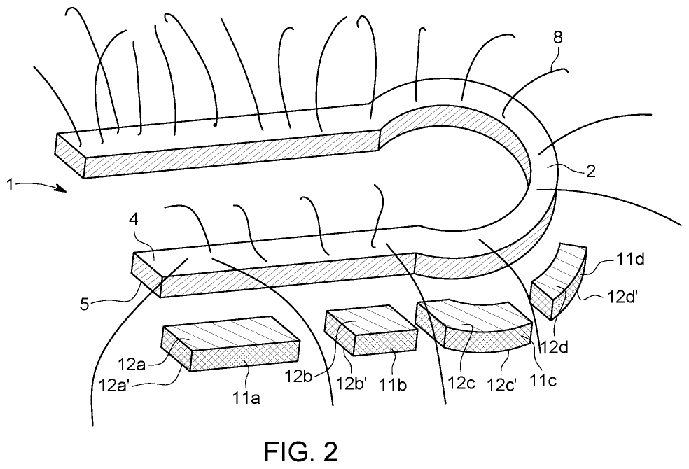

In certain embodiments of the device, as depicted in FIG. 1, the substrate will comprise a single piece substantially identical to the shape of the base. As depicted in FIG. 2, in other embodiments of the device 1, the substrate comprises a plurality of pieces, some of which are depicted as pieces 11a-11d, which when aligned together form a shape substantially identical to the shape of the base 2. The substrate pieces as depicted comprise top and bottom surfaces 12a and 12a′ through 12d and 12d′, respectively. In certain embodiments of the invention, at least one of said base at bottom surface 5 and said substrate pieces at top surfaces, 12a through 12d as depicted, comprise adhesive. In certain embodiments the device of the invention both the bottom surface of the base and top surfaces of the substrate pieces comprise adhesive. Adhesives useful in the practice of the invention are known in the art. Such adhesives include water water-based and solvent-based adhesive systems. Nonlimiting examples of the adhesive include pressure-sensitive adhesives such as, for example, an acrylic pressure-sensitive adhesive such as (meth)acrylic acid ester, a silicone-based pressure-sensitive adhesive such as polyorgano-siloxane, or a urethane-based pressure-sensitive adhesive such as a polyether polyurethane and polyester polyurethane. Other adhesive systems useful in the invention are described in the art, including for example U.S. Pat. Nos. 7,678,959, 6,177,482 and 4,115,549. In certain embodiments the adhesive comprises an aesthetic or functional film which provides tack or grip to a surface of the device. Such films can be adhered to other materials of the base or substrate as part of manufacture of base or substrate materials or applied later. Non-limiting examples of such films include the polyurethane elastic barrier/polyurethane adhesive product known as GRIPTEC sold by Bemis Associates Inc. (MA, USA).

In certain embodiments the adhesives can also comprise a water-based copolymer adhesive, such as those comprising a blend of acetoxyenthylene and dimethylcarbinol. In certain embodiments the adhesives can comprise a solvent-based composition comprising one or more of acrylic based adhesives, isopropanol, and ethylacetate. The adhesive for the present invention may be applied by the wearer or be incorporated as part of the manufacture of the device. When incorporated as part of manufacture, the device of the invention preferably further comprises a removable cover, such as a release strip covering the adhesive, which can be removed prior to installation of the device on the head of the wearer. Adhesives useful herein can comprise a liquid form for application by a brush or similar instrument or a solid form such as a tape which can be applied to the device prior to installation of the device on the head of the wearer.

In certain embodiments, the invention is a reusable device that can be attached, removed and cleaned, and reattached. Removal of the device typically requires a solvent for dissolving the adhesive, in particular a solvent that does not degrade the device. In certain embodiments the solvent can be an alcohol based solvent compatible for use in connection with skin, such as a methyl, ethyl or isopropyl based alcohol or alcohol solution. In other embodiments that solvent can be a water or oil-based solvent or solvent solution for example comprising one or more of water, surfactants, citrus oils, petroleum hydrocarbons, lanolin, mineral oil, and glycols.

In certain embodiments the device of the invention may further comprise a coating layer on bottom surface 5 and top surface 6 that resides between surface 5 and an adhesive layer and surface 6 and an adhesive layer. The coating layer can be applied as part of the manufacturing process or can be a separate coating material that can be applied by the user as desired. Coating layers are useful to protect the surfaces of the device and the device itself from degradation by, for example, the adhesive composition or the solvent release materials. In certain embodiments the coating layer may comprise acrylic compositions, silicones, polyolefins, and methacrylic acid and acrylate ester compositions and the like. In certain embodiments the device of the invention can further comprise a removable and/or reusable sealing mechanism, such as a release tape, that can be applied over a coating layer or an adhesive layer to protect the said layer from degradation before use or between uses of the device. Examples of such removable and/or reusable sealing mechanisms are well known in the art and include materials such as pressure sensitive PET tape, silicone adhesive, silicone rubber, polyester and vinylester materials and the like.

In certain embodiments the invention may comprise an antimicrobial, antifungal or antibacterial agent. In such embodiments, the antimicrobial, antifungal and/or antibacterial agent may be incorporated into one or more of the substrate, base, non-native hair and adhesive. For example, antimicrobial agents can be applied as fabric treatments to the base or substrate materials can be applied during normal textile finishing processes such as by coating, padding, exhaustion or foaming. The antimicrobial agent can typically be used in combination with other common auxiliaries such as softeners, moisture management systems, fluorocarbons or resins. Certain antimicrobial agents for fabrics can also be included during the extrusion process of synthetic fibers such as polyester, polyamide or polypropylene. Other agents can be melt blended into the polymer during processing. In certain embodiments the antimicrobial/antifungal/antibacterial agents are those comprising silver, zinc and/or copper. Other non-limiting examples of agents include zinc pyrithione, silver, silane quaternary ammonia compounds (more commonly known as silane quats), polyhexamethylene biguanide (PHMB), isothiazolinone, thiabendazole, 10,10-oxybisphenoxarsin and 2-n-octy-4ixothiazolin-3-1. Examples of natural antibacterial/antimicrobial/antifungal agents can comprise inorganic acids including carbonates and bicarbonates, or organic acids and their salts, including propionates, sorbates, and benzoates, parabens, chitosan, enzymes, bacteriocins, polypeptides, plant oils, and extracts. Organic acid agents include, but are not limited to, acetic, benzoic, citric, fumaric, lactic, malic, propionic, sorbic, succinic, and tartaric acid. Examples of organic acid salt-based agents include, but are not limited to, potassium sorbate and sodium benzoate.

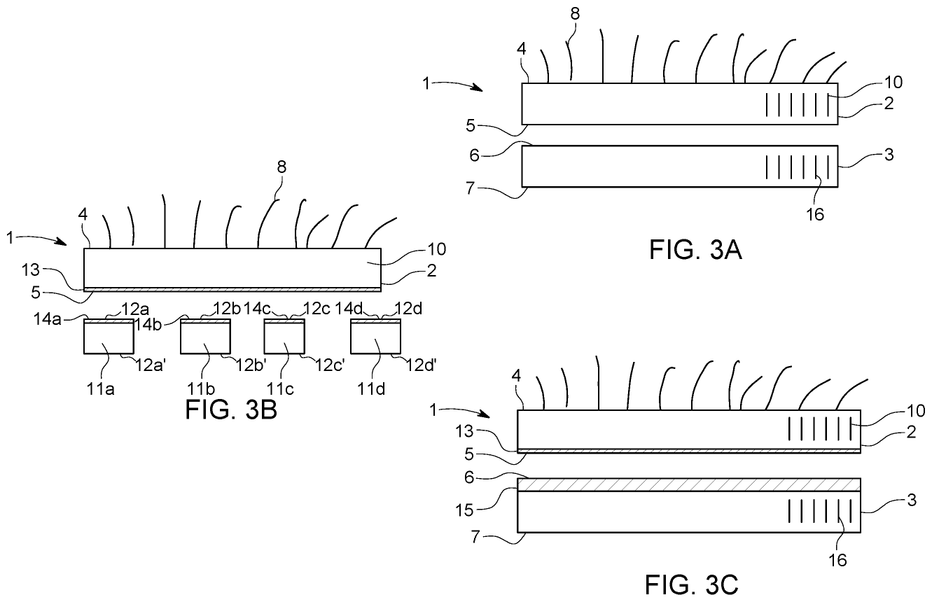

Side views of certain embodiments are depicted in FIGS. 3A-3C, wherein FIG. 3A shows the side view of the device 1 where the base 2 comprising non-native hair 8 and substrate 3 are depicted aligned substantially close to one another showing curved center portion 10 on the base 2 and where substrate 3 comprises a single piece and has a curved center portion 16 corresponding generally in shape to curved center portion 10. FIG. 3B shows the side view of an embodiment of the device wherein the substrate 2 comprising non-native hair 8 is depicted aligned substantially close to the base where the base comprises a plurality of pieces depicted as pieces 11a through 11d, each depicted piece having a top surface and bottom surface, such that piece 11a has top surface 12a and bottom surface 12a′, piece 11b has top surface 12b and bottom surface 12b′, piece 11c has top surface 12c and bottom surface 12c′, piece 11d has top surface 12d and bottom surface 12d′. Those of ordinary skill in the art will recognize that the embodiment shown here may have more or less pieces than depicted in the figure without departing from the scope of the invention. In FIG. 3C the side view of an embodiment the device 1 is shown wherein the substrate 2 comprising non-native hair 8 and base 3 are depicted aligned substantially close to one another showing curved center portion 10 on the substrate 2 and wherein base 3 comprises a single piece and has a curved center portion 16 corresponding generally in shape to curved center portion 10, wherein an adhesive layer 13 is shown applied to the bottom surface 5 of substrate 2 and an adhesive layer 15 is shown applied to the top surface 6 of base 3. Although depicted as a continuous layer of adhesive, the invention does not require a continuous layer but may comprise, for example, a discontinuous layer or spots of adhesive on the respective surfaces. Further, in certain embodiments (not depicted in FIGS. 3A-3C), the device of the invention will comprise an adhesive layer on only one of bottom surface 5 of substrate 2 or top surface 6 of base 3. These embodiments of the invention are useful, for example, where the native hair of the wearer of the device is not present in an amount that would restrict bottom surface 5 of substrate 2 coming in contact with top surface 6 when installed, such that one layer of adhesive on either surface 5 or surface 6 would be sufficient to hold the device in place.

The invention further provides methods of attaching the device to the head of a wearer. Thus in certain embodiments, the invention provides a method of attaching a hair extension device to the native hair on the head of a person in need thereof, wherein the device comprises a substrate, a base and a plurality of nonnative hair pieces attached to said base, and an adhesive composition, said base having a generally crescent shape comprising at least two connected side portions extending from a central region, and said substrate comprising a shape substantially similar to at least a portion of said base, wherein the method comprises aligning the native hair of the person between the base and the substrate and subsequently affixing the base and substrate together by contacting the substrate, base and native hair together with the adhesive, whereby the hair extension device is attached to the native hair on the head of the person.

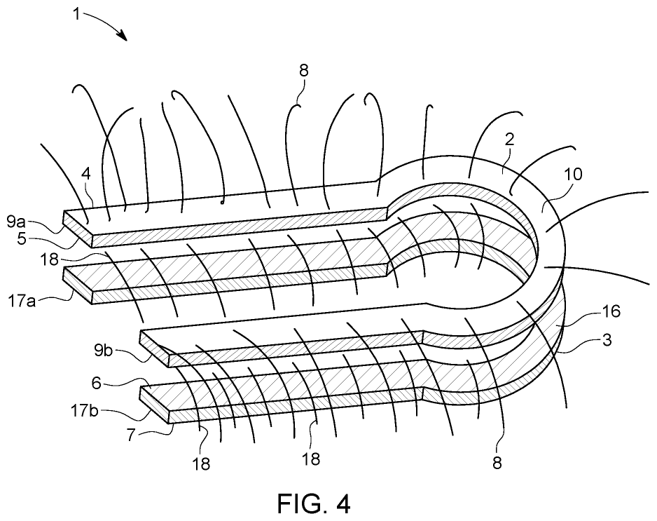

In FIG. 4 a perspective view of an embodiment of device 1 is shown comprising base 2 and substrate 3, said base comprising a top surface 4 and a bottom surface 5 and a plurality of nonnative hair pieces 8 secured thereto and extending outward from top surface 4. Base 2 comprises a generally crescent shape comprising a center portion 10 and at least two side portions 9a and 9b extending outward from said center portion 10, said side portions depicted as aligned generally parallel to one another as would be seen when attached to the head of a person. In the embodiment shown, substrate 3, having top surface 6 and bottom surface 7, comprises a single piece similar in shape to base 2 and a corresponding center portion 16 and at least two side portions 17a and 17b extending outward from said center portion 16, said side portions depicted as aligned generally parallel to one another as would be seen when attached to the head of a person. Device 1 is shown in FIG. 4 where the native hair of the wearer 18 is depicted placed between the top surface 6 of substrate 3 and the bottom surface 5 of base 2 and integrated with the non-native hair 8 attached to the top surface 4 of base 2. In certain embodiments (not depicted in FIG. 4) adhesive applied to one or both of top surface 6 of substrate 3 and bottom surface 5 of base 2 contact native hair 18 and hold the device 1 in place on the head of the wearer when base 2 and substrate 3 are contacted together in a manner that sandwiches the adhesive and native hair between base 2 and substrate 3, which are then substantially immobilized in place on the head of the wearer as a result of the adhesive. Such contacting can comprise merely the pressure of fingers or hands compressing base 2 and substrate 3 together or the use of a crimping tool or other similar pressure delivering instrument. The non-native hair 8 of the device can then be integrated with native hair 18, by hand or instrument, such as a brush or comb or other instrument known to those of ordinary skill in the art.

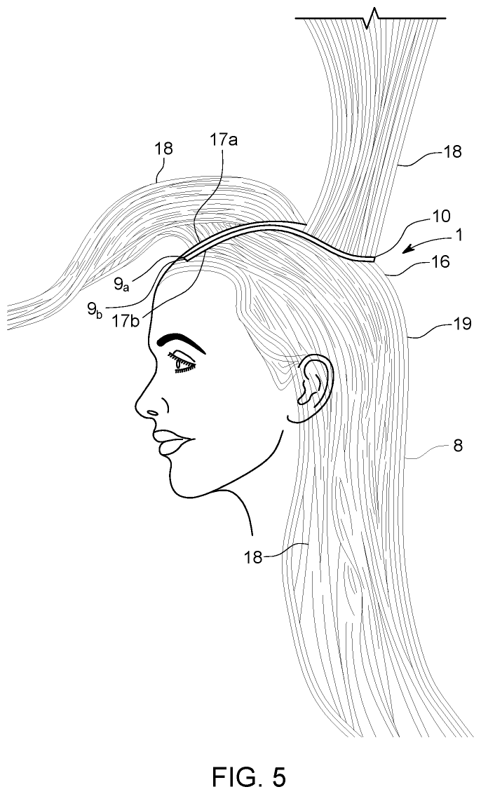

FIG. 5 shows a side view of an embodiment of the device 1 as installed on the head of a wearer 19, demonstrating also an embodiment of a method of the invention. The device 1 is mounted on the wearer's native hair 18 and side pieces 9a and 9b of the substrate and 17a and 17b of the base are aligned substantially parallel to one another at a portion near the top of the wearer's head. Additional native hair 18 is aligned through the openings created by the space between the side pieces 9a and 9b and 17a and 17b as well as through the curvature of center portions 10 and 16. According to the methods of the invention, the native hair 18 is then overlaid atop the device 1 as installed and then integrated with the non-native hair 8 that is connected to device 1.

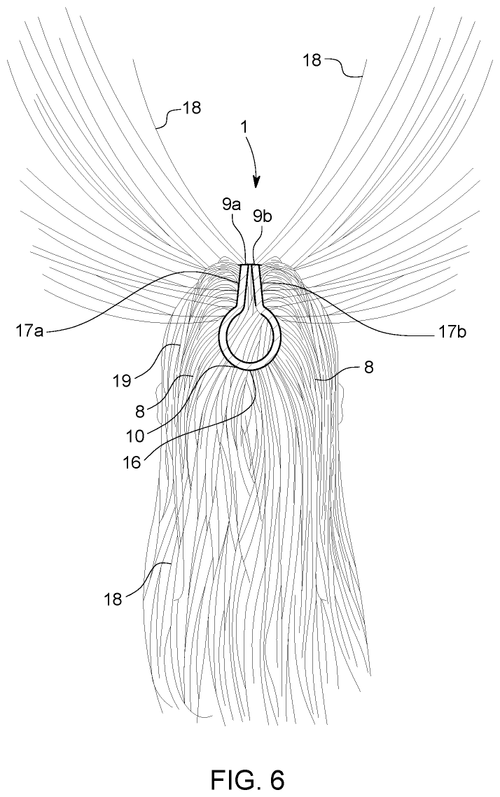

FIG. 6 is a depiction of an embodiment of the device 1 as installed on the head of a wearer 19 from a perspective of the top rear of the wearer's head, demonstrating also an embodiment of a method of the invention. The device 1 is mounted on the wearer's native hair 18 and side pieces 9a and 9b of the substrate and 17a and 17b of the base are aligned substantially parallel to one another at a portion near the top of the wearer's head. Additional native hair 18 is aligned through the openings created by the space between the side pieces 9a and 9b and 17a and 17b as well as through the curvature of center portions 10 and 16. According to the methods of the invention, the native hair 18 is then overlaid atop the device 1 as installed and then integrated with the non-native hair 8 that is connected to device 1.

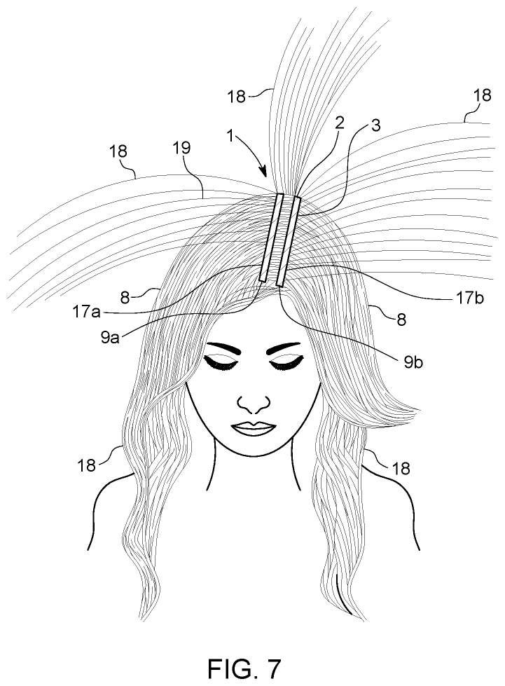

FIG. 7 is a depiction of an embodiment of the device 1 as installed on the head of a wearer 19 from a perspective of the top front of the wearer's head, demonstrating also an embodiment of a method of the invention. The device 1 is mounted on the wearer's native hair 18 and side pieces 9a and 9b of the substrate and 17a and 17b of the base are aligned substantially parallel to one another at a portion near the top of the wearer's head. As depicted in FIG. 7, the device 1 is aligned at a portion of the head off-center of the top of the wearer's head. Additional native hair 18 is aligned through the openings created by the space between the side pieces 9a and 9b and 17a and 17b as well as through the curvature of center portions 10 and 16. According to the methods of the invention, the native hair 18 is then overlaid atop the device 1 as installed and then integrated with the non-native hair 8 that is connected to device 1.

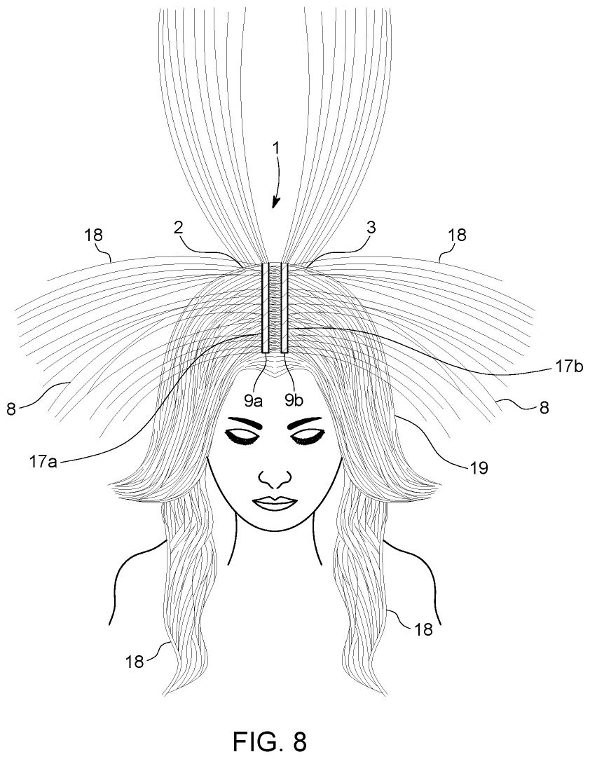

FIG. 8 is a depiction of an embodiment of the device 1 as installed on the head of a wearer 19 from a perspective of the top front of the wearer's head, demonstrating also an embodiment of a method of the invention. The device 1 is mounted on the wearer's native hair 18 and side pieces 9a and 9b of the substrate and 17a and 17b of the base are aligned substantially parallel to one another at a portion near the top of the wearer's head. As depicted in FIG. 7, the device 1 is aligned at a portion of the head at a portion approximating the center of the top of the wearer's head. Those of ordinary skill in the art will recognize that actual final alignment of device 1 at or near the center of the head is done according to such factors as the amount of native hair at the top of the head, the topography of the wearer's head and needs of the wearer and any such slight deviations in alignment does not depart from the scope of the invention. Additional native hair 18 is aligned through the openings created by the space between the side pieces 9a and 9b and 17a and 17b as well as through the curvature of center portions 10 and 16. According to the methods of the invention, the native hair 18 is then overlaid atop the device 1 as installed and then integrated with the non-native hair 8 that is connected to device 1.

The invention further provides a kit for use in attaching a hair extension device of the invention to the native hair on the head of a person in need thereof. In certain embodiments, the kit comprises a hair extension device of the invention, such as a device comprising a substrate, a base and a plurality of nonnative hair pieces attached to said substrate, said substrate having a generally crescent shape comprising at least two connected side portions extending outward from each other, and said base comprising a shape substantially similar to said substrate. In certain embodiments, the kit comprises a package to hold all the components of the kit. In certain embodiments, the kit further comprises instructions for attaching the hair extension device to the native hair on the head of the person. In certain embodiments, the kit comprises a device having a substrate and/or base comprising an adhesive material. In certain embodiments, the kit comprises an adhesive material as a separate object. The adhesive materials may be any of the adhesives or combinations thereof as described above. In certain embodiments, the kit further comprises tools for application of the adhesive to the device. In certain embodiments, the kit further comprises tools for manipulating the device to facilitate installation of the device, such as for example to hold the device in place on the head of the wearer during installation or to provide means to apply pressure to attach the base and/or substrate to one another or to the native hair.

### Example

A hair extension device was prepared by attaching human hair strands to a base comprising a woven fabric comprising 40/24 DL Polyester and 140 Spandex. Prior to application of the hair the woven fabric of the base, the woven fabric was heat pressed with a polyurethane adhesive film material GRIPTEC ET315 (BEMIS) to form a woven fabric with a grip surface. The non-native hair was attached by hand and knotted to the woven fabric. The grip surface was on the bottom surface of the base, facing away from the non-native hair. Additional film material was then added after the attachment of the non-native hair. The base comprising the gripped woven fabric material was shaped in a crescent shape or keyhole shape as described herein. The substrate and base materials comprised the same woven fabric material. Both substrate and base were single pieces substantially similarly crescent/keyhole shaped. A water-based adhesive was applied to the top surface of the substrate and the bottom surface of the base. In the installation of the device no adhesive is applied to the scalp of the head.

The device was installed on a mannequin head which is designed to display female pattern baldness (decreased amount of hair at the center top portion of the head and increasing amount of hair along the sides of head). The device was installed substrate first, applied to native hair near the top of the head, aligning the elongated pieces along the top of the head in order to facilitate formation of a hair part on the top of the head when the device is fully installed. Before the substrate was installed a half inch thickness of native hair was sectioned off and secured away from where the device was to be installed. Next, an eighth of an inch of the native hair was applied over the adhesive top layer on the substrate and then the base was applied over the top of the native hair, pressing the base and substrate together to adhere the two adhesive layers together, sandwiching the native hair in between the base and substrate and thus securing the device to the head. The remaining sectioned off native hair was then laid over the top of the substrate containing the non-native hair and the native and non-native hair were intermingled by hand, comb and brush to evenly disperse the native and non-native hair on the head of the mannequin forming a natural looking part at the top of the head.

After installation, the native and non-native hair was worked into various hair styles including up, down, and pulled back styles. The device stayed in place and allowed the non-native hair to be manipulated seamlessly with the native hair, giving the impression of a full head of native hair able to be worked into various normal hair styles while maintaining a natural looking part at the top of the head. After the hair styling, the device was removed by applying rubbing alcohol to the device to dissolve the water-based adhesive. Once separated from the native hair the device was washed with normal shampoo and dried for re-use.

While the invention has been described with respect to certain non-limiting embodiments above, it is intended to fully cover such alternatives, modifications and equivalents as included within the scope of the invention defined by the claims appended hereto.

1. A removable device for temporary attachment to the head of a person having native hair, said device comprising a base and a substrate, said base comprising a top surface and a bottom surface and a plurality of nonnative hair pieces secured thereto and extending outward from said top surface, said base having a generally crescent shape comprising a center portion and at least two side portions extending outward from said center portion, said side portions aligned generally parallel to one another when attached to the head of a person, said substrate comprising a top and a bottom surface and having a shape similar to at least a portion of the shape of said base, and at least one of said bottom surface of said base and said top surface of said substrate adapted to receive an adhesive.

2. The device of claim 1, wherein the substrate has a shape comprising a single piece substantially identical to the shape of the base.

3. The device of claim 1, wherein the substrate comprises a plurality pieces, which when aligned together form a shape substantially identical to the shape of the base.

4. The device of claim 1, wherein at least one of said base and said substrate comprises an adhesive.

5. The device of claim 1, wherein both the base and substrate comprise adhesive.

6. (canceled)

7. A method of attaching a hair extension device to the native hair on the head of a person in need thereof, wherein the device comprises a substrate, a base and a plurality of nonnative hair pieces attached to said base, and an adhesive composition, said base having a generally crescent shape comprising at least two connected side portions extending from a central region, and said substrate comprising a shape substantially similar to at least a portion of said base, wherein the method comprises aligning the native hair of the person between the base and the substrate and subsequently affixing the base and substrate together by contacting the substrate, base and native hair together with the adhesive, whereby the hair extension device is attached to the native hair on the head of the person.

8. The method of claim 7, wherein the device is attached to the center of the top of the head of a person.

9. (canceled)

10. The method of claim 7, wherein adhesive is applied to at least one of said base and said substrate just prior to the step of aligning the native hair of the person between the base and the substrate.

11. The method of claim 7, wherein the adhesive is applied to the native hair at a portion that will be contacted by at least one of the substrate and the base.

12. (canceled)

13. (canceled)

14. (canceled)

15. A kit for use in attaching a hair extension device to the native hair on the head of a person in need thereof, wherein the kit comprises a hair extension device, said device comprises a substrate, a base and a plurality of nonnative hair pieces attached to said substrate, said substrate having a generally crescent shape comprising at least two connected side portions extending outward from each other, and said base comprising a shape substantially similar to said substrate.

16. The kit of claim 15, wherein at least one of said substrate and said base comprises an adhesive.

17. The kit of claim 15, further comprising an adhesive for application to said hair extension device.

18. The kit of claim 15, further comprising instructions for attaching the hair extension device to the native hair on the head of the person.

19. The device of claim 1, wherein at least one of said base and said substrate comprise a woven fabric.

20. The device of claim 4, wherein the adhesive comprises a water-based copolymer adhesive.

21. The device of claim 20, wherein the water-based copolymer adhesive is chosen from the group consisting of acetoxyenthylene, dimethylcarbinol and mixtures thereof.

22. The device of claim 4, wherein the adhesive comprises a solvent-based adhesive.

23. The device of claim 22, wherein the solvent-based adhesive is chosen from the group consisting of acrylic based adhesives, isopropanol, ethylacetate and mixtures thereof.

24. The device of claim 4, wherein the adhesive comprises an aesthetic or functional film which provides tack or grip to a surface of the device.

25. The device of claim 24, wherein the aesthetic or functional film is a polyurethane elastic barrier/polyurethane adhesive.
